(19) United States
(12) Patent Application Publication
(10) Pub. No.: US 2018/0368938 A1
(43) Pub. Date: Dec. 27, 2018

(54) DENTAL LIGHT

(71) Applicant: Microcopy Ltd., Afula (IL)

(72) Inventors: Perry Parke, Marietta, GA (US); Emily Brayman, Atlanta, GA (US); Jeremy Gura, Peachtree City, GA (US); Brian Kim, Atlanta, GA (US); Alvin Laohapant, Silver Spring, MD (US); Jorge E. Perdomo, Miami, FL (US)

(21) Appl. No.: 15/961,623

(22) Filed: Apr. 24, 2018

Related U.S. Application Data
(60) Provisional application No. 62/489,084, filed on Apr. 24, 2017.

(51) Int. Cl. A61C 1/08

(52) U.S. Cl. CPC A61C 1/088; A61B 1/24

(57) ABSTRACT

A dental light (10/60) is disclosed having a housing (11/61) with a first leg (13/63), a second leg (14/64), and a middle portion (16/66). The first leg includes a light chamber (21/71) configured to receive and hold a small light source (22/72). The second leg includes a battery compartment (26/76) configured to house a battery source (29/79). The dental light is coupled to a patient's mouth, specifically the patient's lip, with the first leg residing within the interior of the mouth and the second leg position outside the mouth. The patient's lip should reside comfortably within the space (38/88) between the first and second legs.

### REFERENCE TO RELATED APPLICATION

Applicant claims the benefit of U.S. Provisional Patent Application Ser. No. 62/489,084 filed Apr. 24, 2017 and entitled Dental Light.

### TECHNICAL FIELD

This invention relates generally to dental lights which are used in the dental industry.

### BACKGROUND OF THE INVENTION

Lights which are used in the dental industry have typically been large overhead lights mounted to the ceiling of a dental office. These lights use large reticulating arms to position the light in a position to illuminate the mouth of a patient. However, because the dentist is working between the patient and the overhead light, the illumination produced by the overhead light is blocked by the dentist or the instruments being used by the dentist, i.e., a shadow is cast upon the mouth of the patient.

To overcome the problem with creating a shadow, some dental lights have been designed to fit within a patient's mouth. Some such lights have taken on the form of a dental retractor which includes a light source, such as that shown in U.S. Pat. No. 4,592,344 and U.S. Patent Publication No. 2012/0228528. While these devices provide a light source, they are cumbersome and oftentimes causes discomfort to the patient. Similarly, some lights have taken on the form of a bite spacer, as shown in U.S. Pat. Nos. 6,022,214 and 9,532,858 and U.S. Patent Publication Nos. 2005/0239018; 2006/0269898; and 2008/0032252. Again, these devices are cumbersome and oftentimes causes discomfort to the patient. Lastly, a dental light has been designed to be bent and hung upon a patient's lip, such as that shown in U.S. Pat. No. 6,304,712. A problem associated with these types of lights is that they utilize ancillary portions, such as power cords or light bars, therefore the weight of the light upon the patient's lip causes discomfort. Furthermore, they cannot easily illuminate the upper portions of the mouth.

Accordingly, it is seen that a need remains for a dental light which may be easily used and manipulated while also being comfortable for the patient It is to the provision of such that the present invention is primarily directed.

### BRIEF DESCRIPTION OF THE DRAWING

FIG. 1 is a perspective view of a dental light in a preferred form of the invention.

FIG. 2 is a perspective view of the dental light of FIG. 1.

FIG. 3 is side view of a portion of the dental light of FIG. 1.

FIG. 4 is partial view of a spring component portion of a dental light in another preferred from of the invention.

FIG. 5 is a perspective view of a dental light in another preferred form of the invention.

FIG. 6 is a perspective view of a dental light in another preferred form of the invention, shown in a straight configuration.

FIG. 7 is a side view of the dental light of FIG. 6, shown in a bent configuration.

FIG. 8 is a top view of the dental light of FIG. 6, showing internal components in phantom lines.

FIG. 9 is a wiring diagram of the dental light of FIG. 1.

### DETAILED DESCRIPTION

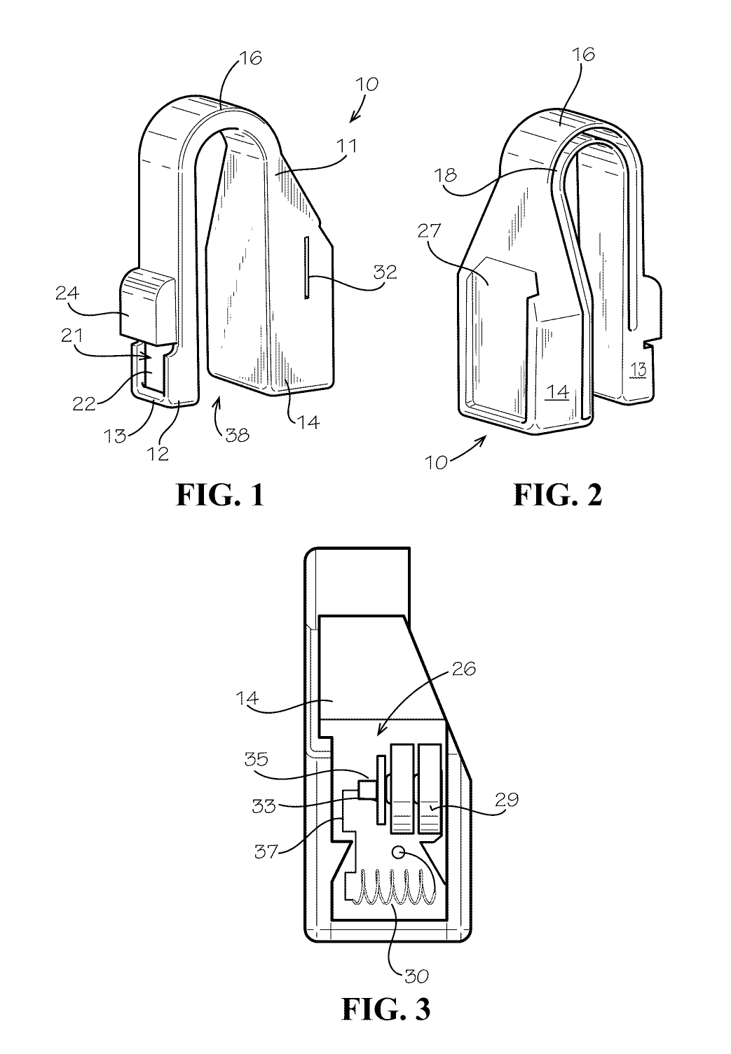

With reference next to the drawings, there is shown in FIGS. 1-4 a self-contained dental light 10 embodying principles of the invention in a preferred form. The dental light 10 has a U-shaped housing 11 having a first portion or leg 13, a second portion or leg 14, and a U-shaped middle, bent, bight or bight portion 16 extending between the first and second legs 13 and 14. The housing 11 includes an internal trough or slot 18 which extends through the first leg 13, bight 16 and second leg 14. The housing 11 is preferably made of a plastic material or the like.

The first leg 13 includes a light recess or chamber 21 configured to receive and hold a small light source 22, such as one or more LED lights, to protect the LED from direct contact with the patient, and visa-versa. The first leg 13 also includes a light deflector 24 mounted directly above the light source 22. The light deflector 24 is intended to prevent light from shining in the dentist's eyes while the dental light 10 is in use within a patient's mouth.

The second leg 14 includes a battery compartment 26 having a door 27 for accessing the interior of the battery compartment 26. The battery compartment 26 is configured to house a battery source 29 and a resistor 30, such as the two batteries shown in FIG. 3. The basic electrical circuit of a battery source 29 and resistor 30 may be expanded to include other conventional types of electrical components if a more controllable light circuit is desired.

The battery compartment 26 includes a battery blocker slot 32 which releasably receives a non-conductive battery blocker or blocker slip 33 which is initially positioned between the battery source 29 and a battery contact 35 to prevent the completion of a closed circuit, i.e., prevent the battery from being in electrical contact with the battery contact 35. The battery blocker slip 33 is removed from between the battery source 29 and contact 35 to activate the dental light 10. Electrically conductive wires 37 extend between the battery contacts 35, resistor 30, and light source 22 to power the light source 22. The electrically conductive wires 37 are positioned within the trough 18 for easy mounting during manufacturing. It should be understood that the configuration shown here is intended to be a one time use or disposable type light, however, should a reusable light be desired the control circuit would include an on/off switch rather than the blocker slip 33 described herein. If made reusable, the batteries may be removable from the housing.

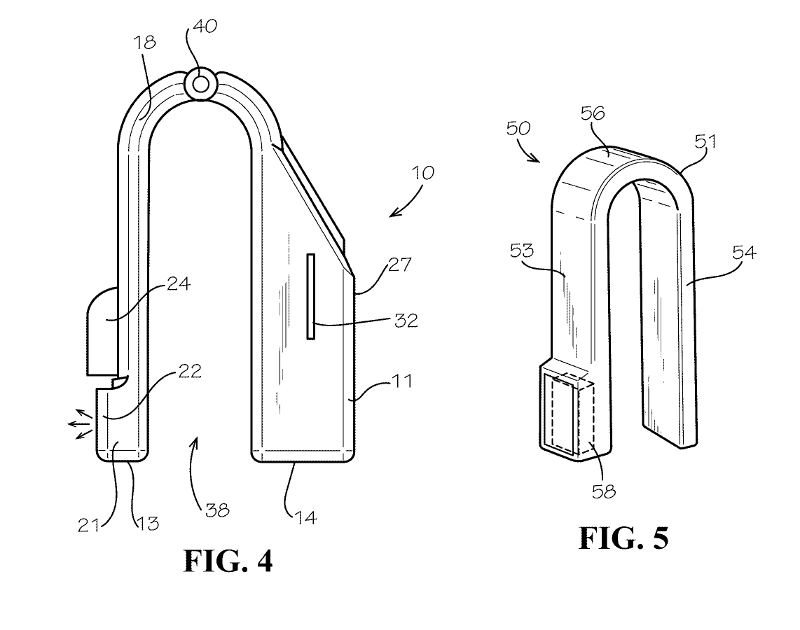

The U-shaped bight portion 16 of the light 10 may be generally rigid, unbending, inflexible if the lip holding space 38 between the first and second legs 13 and 14 is intended to be relatively stable. Alternatively, the U-shaped bight portion 16 may be flexible or semi-flexible to provide a spring biased action to produce a slight pinching of the lip or lip area during use. As another alternative, the U-shaped bight portion 16 may include a spring 40, as shown in FIG. 4, to provide a spring biasing action to slightly pinch or hold the lip during use.

The entire housing 11 may be coated with a soft plastic or rubber material to provide a more comfortable feel for the patient and insulate the circuit.

In use, the dental light 10 is coupled to a patient's mouth, specifically the patient's lip, with the first leg 13 residing within the interior of the mouth and the second leg 14 position outside the mouth. The patient's lip should reside comfortably within the space 38 between the first and second legs 13 and 14. The second leg 13, containing the bulkier battery and electronics components, is positioned outside the mouth to minimize the volume within the mouth for maximum comfort and maintain patient safety as to not accidentally bite on electrical components or the battery.

With the light 10 in this position, the light emanating from the light source 22 illuminates the interior of the patient's mouth. Because of the dental light's small configuration and light weight construction, the patient does not feel any, or at least very little, discomfort associated with the dental light 10. Also, the fight does not interfere or contact the teeth, thus providing a better ability to work upon the teeth. The supple nature of a person's lip allows the dental light 10 to be held in place even if the patient's head is subsequently moved to a different position. However, the optional spring biasing of the U-shaped bight portion 16 may aid the holding of the dental light. Obviously, the dental light 10 may be moved to different locations along the patient's lip during use if a different light illumination or direction of illumination is desired by the dentist.

With reference next to FIG. 5, there is shown a dental light 50 in another preferred form of the invention. Here, the dental light 50 includes a U-shaped housing 51 having a first leg 53, second leg 54 and a U-shaped bight portion 56 therebetween. However, here the first leg 53 includes a fluid compartment 58 therein which is designed to store an illumination chemicals, which reacts to glow when combined, i.e., the light source is a phosphorous material or the like. Such a phosphorous material may be a dibutyl phthalate, or a small glass vial inside the plastic tube that contains a mixture of hydrogen peroxide in phthalic ester. The fluid compartment 58 may include a transparent or translucent window or may simply house a transparent or translucent vial containing the glowable chemicals.

It should be understood that the U-shape housing is a general shape and the legs are not required to be of the same length. The housing may also be generally J-shaped, a hook shape, be more squared off rather than round, or may even be flat and then manually contoured by the dentist into a desired shape which preferably included a hook or crook to hold onto the lip of the patient. Thus, U-shaped is intended to denote a shape wherein a portion is bent so that two portions generally overlay each other.

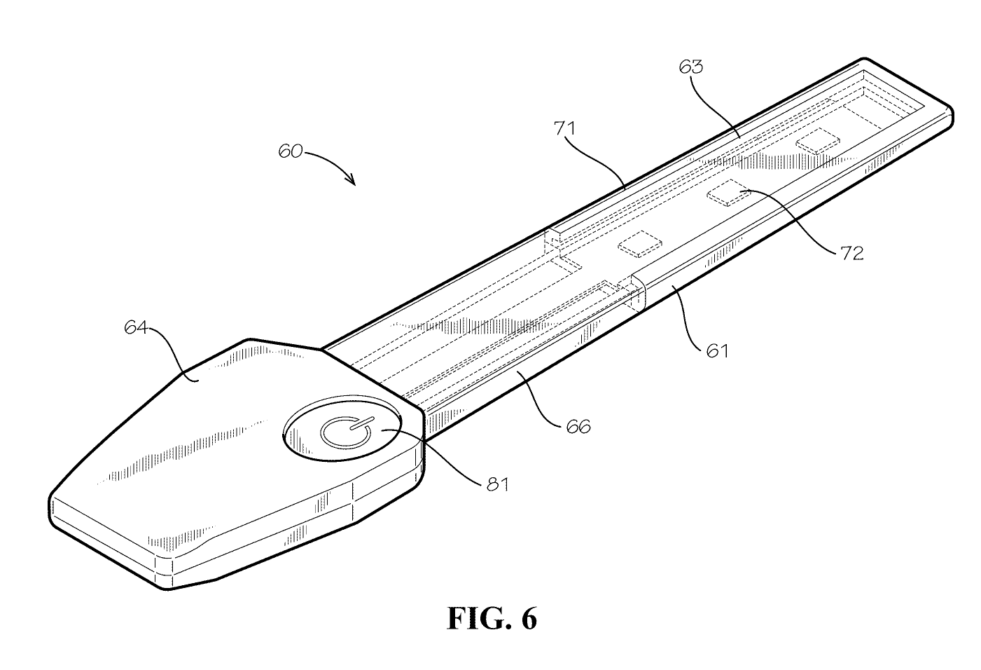

With reference next to the drawings, there is shown in FIGS. 6-9 a dental light 60 embodying principles of the invention in another preferred form. The dental light 60 has a generally straight, initially shaped housing 61 having a first portion or leg 63, a second portion or leg 64, and a bendable or pliable middle, bridge or bight portion 66 extending between the first and second legs 63 and 64. The housing 11 is preferably made of a plastic material or the like. The bight portion 66 may include an embedded strip of metal 67 which enables the bight portion to retain its bent shape. The term pliable as used herein is intended to mean a material or object which may be manually bent and wherein the material generally retains the bent shape once released.

The first kg 63 includes a light chamber 71 configured to receive and hold a small light source 72, shown as a set of three LED lights.

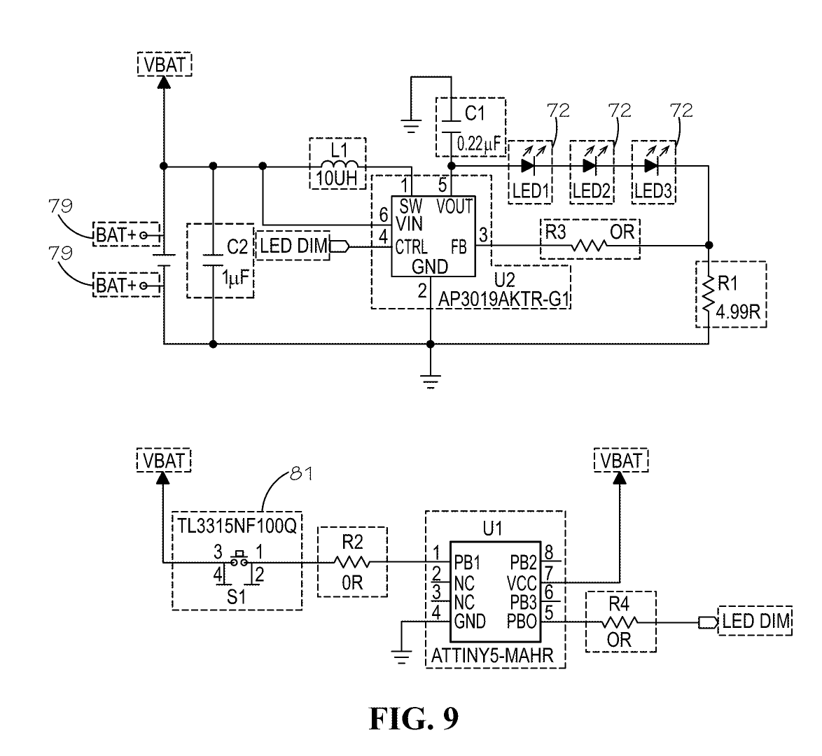

The second leg 64 includes a battery compartment 76 configured to house a battery source 79, a circuit board 80, and an on/off switch 81. FIG. 9 illustrates the basic electrical circuit of a battery source 79, circuit board 80 and on/off switch 81 may be expanded to include other conventional types of electrical components if a more controllable light circuit is desired. The circuit may also be configured to vary the brightness or intensity of the light emitted from the dental light through the on/off switch.

As an alternative to having the on/off switch 81, this embodiment may be adapted to include the previously described battery blocker slot and non-conductive battery blocker or blocker slip. Electrically conductive wires extend between the circuit board 80 and the light source 72 to power the light source 72. As another alternative, the activation of the light may be actuated by the bending of the bight portion 66.

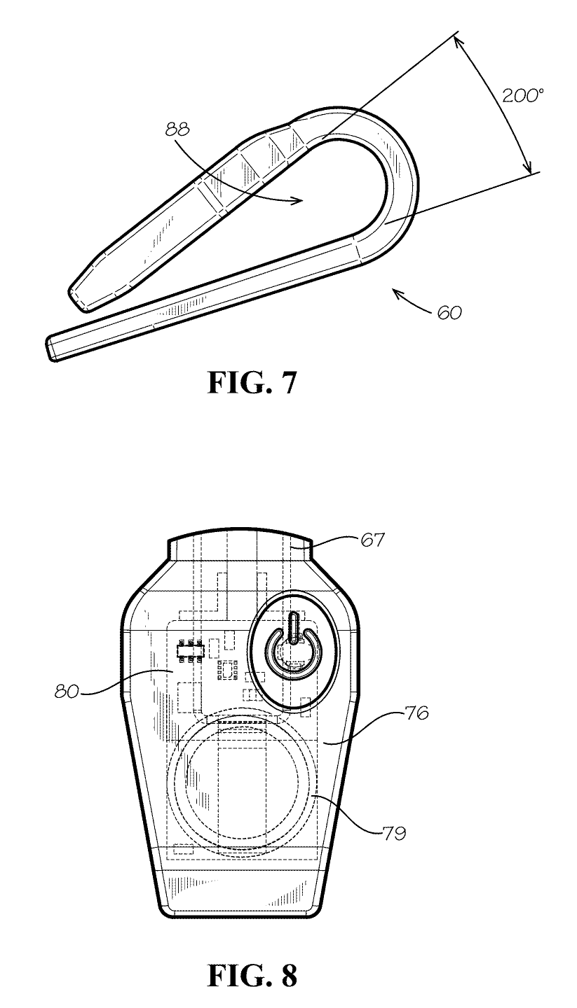

The bight portion 66 of the light 60 is made of a pliable material which holds its shape when bent, as shown in FIG. 7, so as to form a lip holding space 88 between the first and second legs 63 and 64 is intended to be relatively stable. The light 60 may be used in its first or straight configuration in some dental applications. An angle of 200 degrees is shown, however, other angles which are less or greater are also possible.

FIG. 9 illustrates an example of a circuit utilized for the dental light 60.

In use, the light 60 may be utilized by simply bending the bight portion 66 about the lip of a patient so as to position the light source 72 at the proper position to illuminate the intended work area of the patient's mouth.

It should be understood that the number and arrangement of lights or light emitting diodes (LEDs) may be varied without departure from the scope of the invention. The number and arrangement or configuration of lights may be varied to provide different brightnesses or areas of illumination.

It should also be understood that the light may include an orthodontic specific filter, such as an orange light filter to prevent the premature hardening of select dental adhesives or materials. The light source may also be that of a specific wavelength to aid in the detection of cancers, micro-cavities or other dental related problems. Also, the light source may be in the form of a translucent housing that illuminates an entire area.

It should also be understood that the dental lights shown herein are entirely self-contained, thus, there are no wires or other portions which cause extra weight or otherwise may limit movement of the light or pull upon a patient's mouth, such as that shown in U.S. Pat. No. 6,304,712. Thus, self-contained is intended to mean a dental light which does not have any power cords, accessory wires, light tubes, or any other ancillary portion.

The dental light may be configured so that the leg 13 or 63 is in the form of a bite block to maintain the mouth in an open position.

Lastly, the second leg 14 or 64 may be removable so that this component is reusable.

It thus is seen that a dental light is now provided which is more comfortable for a patient and without interfering with the viewing of the teeth. While this invention has been described in detail with particular references to the preferred embodiments thereof, it should be understood that many modifications, additions and deletions, in addition to those expressly recited, may be made thereto without departure from the spirit and scope of the invention as described by the following claims.

1. A dental light comprising,
a first portion having a light source;
a second portion having a battery source electrically coupled to said light source of said first portion, and
a U-shaped bight portion extending between said first portion and said second portion, said U-shaped bight portion forming a channel sized and shaped to hold a person's lip area.

2. The dental light of claim 1 wherein said bight portion includes a spring biasing said first portion and said second portion towards each other.

3. The dental light of claim 1 wherein said first portion includes a deflector to block light from said light source in a direction towards said bight portion.

4. The dental light of claim 1 wherein said U-shaped bight portion is made of a bendable material adapted to be converted from a straight configuration to a U-shaped configuration.

5. The denial light of claim 1 wherein said housing first portion includes a recess and wherein said light source is positioned within said recess.

6. A dental light comprising a housing having a light source and a battery source electrically coupled to said light source, said housing having a U-shaped configuration defining a lip channel configured to receive and hold a person's lip.

7. The dental light of claim 6 wherein said housing has a first portion coupled to said light source, a second portion coupled to said battery source, and a bight portion extending between said first portion and said second portion.

8. The dental light of claim 7 wherein said bight portion includes a spring biasing said first portion and said second portion towards each other.

9. The dental light of claim 7 wherein said first portion includes a deflector to block light from said light source in a direction towards said bight portion.

10. The dental light of claim 7 wherein said bight portion is made of a bendable material adapted to be converted from a straight configuration to a U-shaped configuration.

11. The dental light of claim 6 wherein said housing includes a light recess and wherein said light source is positioned within said light recess.

12. A self-contained dental light adapted to be used within the mouth of a dental patient, the self contained dental light comprising,
a housing having a first portion, a second portion and a middle portion extending between said first portion and said second portion, said middle portion being made of a pliable material;
a light source coupled to said housing, and
a battery source electrically coupled to said housing.

13. The self-contained dental light of claim 12 wherein said housing middle portion is pliable between a first configuration wherein said middle portion is straight and a second configuration wherein said middle portion is U-shaped so as to define a lip channel configured to receive and hold a person's lip.

14. The self-contained dental light of claim 12 wherein said light source is coupled to said housing first portion.

15. The dental light of claim 14 wherein said housing first portion includes a recess and wherein said light source is positioned within said recess.

16. The self-contained dental light of claim 14 wherein said battery source is coupled to said housing second portion.

17. A self-contained dental light adapted to be used within the mouth of a dental patient, the self contained dental light comprising,
a housing having a first portion, a second portion and a middle portion extending between said first portion and said second portion, said middle portion being made of a pliable material, and
a light source coupled to said housing.

18. The self-contained dental light of claim 17 wherein said light source is a phosphorous material.

19. The self-contained dental light of claim 17 wherein said housing middle portion is pliable between a first configuration wherein said middle portion is straight and a second configuration wherein said middle portion is U-shaped so as to define a lip channel configured to receive and hold a person's lip.

20. The dental light of claim 17 wherein said housing first portion includes a recess and wherein said light source is positioned within said recess.
